(19) United States
(12) Patent Application Publication
(10) Pub. No.: US 2019/0083316 A1
(43) Pub. Date: Mar. 21, 2019

(54) WELDING MASK WITH SENSOR LAMP

(71) Applicant: Ningbo Geostar Photoelectric Technology Co., Ltd, Ningbo High-tech Zone (CN)

(72) Inventors: Zhaolin YANG, Ningbo High-tech Zone (CN); Jianrong ZHOU, Ningbo High-tech Zone (CN); Rongjiang WANG, Ningbo High-tech Zone (CN)

(73) Assignee: Ningbo Geostar Photoelectric Technology Co., Ltd, Ningbo High-tech Zone (CN)

(21) Appl. No.: 15/856,308

(22) Filed: Dec. 28, 2017

(30) Foreign Application Priority Data
Sep. 18, 2017 (CN) 201721194789.4

(51) Int. Cl. A61F 9/06; F21V 23/04; F21L 4/08

(52) U.S. Cl. CPC A61F 9/065; F21L 4/08; F21V 23/04; F21V 23/0464

(57) ABSTRACT

A welding mask with a sensor lamp provides a welding protector. The welding mask with the sensor lamp includes a mask shell covering a human face, a filter module arranged on the mask shell, a head lamp arranged on the filter module, and a control switch connected to the cord of the head lamp, and the control switch comprises a mechanical switch and a sensing module; the head lamp is provided on the filter module according to the present disclosure, so that lighting will be provided for operator in poor lighting environment, then the welding quality can be guaranteed; the mechanical switch and the sensing module, and the mechanical switch has a plurality of gear selections, whereby selection in relation to normally-on, normally-off and sensor circuit is achieved, which boasts higher security.

### CROSS-REFERENCE TO RELATED APPLICATIONS

The present application claims priority to and the benefit of Chinese Patent Application No. CN 201721194789.4 filed on Sep. 18, 2017, the entire contents of which are incorporated herein fully by reference.

### BACKGROUND OF THE DISCLOSURE

### 1. Field of the Disclosure

The present disclosure generally relates to the technical field of welding protector, and more particularly, to a welding mask with a sensor lamp.

### 2. Description of the Related Art

Weldment has been widely used in all walks of life. Since performance of the weldment is more reliable than that of a connector, and the weldment performs better in terms of utilization rate, it is important to guarantee the quality of the weldment.

At present, owing to different working conditions, in the case of bright light, the weldment can be seen clearly during welding to complete the welding operation. However, in the case of poor environment with weak light, it is barely possible for an operator to observe the weldment using his eyes only, and in such an environment with poor lighting, welding quality cannot be properly guaranteed, then it is thus desirable to develop a welding protector with a head lamp, especially a welding mask with the head lamp.

However, there are two types of welding masks with the head lamp available in the market: one is a chuck-typed, that is, the head lamp is gripped by a chuck whereby the head lamp is arranged on the welding mask, and switch-on and switch-off is controlled by an external switch; and the other is welding the head lamp on the welding mask directly, and switch-on and switch-off is done manually. However, problems listed as follows exist in those two types of welding masks. Too many components involved in these two types of welding masks, it is inconvenient to carry with, or manual operation is necessary for the control of the head lamp. In addition, during welding, since welding arc can compensate for the lack of light, it is a waste of electric energy if the head lamp is still on then.

### SUMMARY OF THE DISCLOSURE

Given that the foregoing problems exist in the prior art, the present disclosure provides a welding mask with a sensor lamp, that is, a head lamp is arranged on the component where lens of the welding mask is located, and is connected to a light sensing sensor, which will be switched on automatically in a weak light, while switched off automatically in an environment with bright light, whereby a modular head lamp is achieved, and electric energy can be saved a lot.

The specific technical schemes are as follows:

A welding mask with a sensor lamp, comprising: a mask shell, a filter module, a head lamp and a control switch, wherein the filter module is arranged on the mask shell, the head lamp is embedded in the filter module and is connected to a circuit board of the filter module; wherein,

the control switch comprises a mechanical switch and a sensing module, and both the mechanical switch and the sensing module are connected to the circuit board of the filter module.

The above welding mask with a sensor lamp, wherein the mask shell comprises a face portion and two side portions, and the face portion is just in front of eyes of an operator when in use, while the two side portions are disposed on either side of the face portion respectively.

The above welding mask with a sensor lamp, wherein the face of the mask shell is provided with a filter window, and the filter window is rectangular.

The above welding mask with a sensor lamp, wherein the mechanical switch is provided with a plurality of gears, one of the plurality of gears is a normally-off gear and another one of the plurality of gears is a normally-on gear, and another one of the plurality of gears is a sensing gear, and the mechanical switch in the sensing gear is connected to the sensing module.

The above welding mask with a sensor lamp, wherein the sensing module is embedded in the filter module, and the sensing module further comprises a light sensing sensor and a corresponding control circuit, wherein the light sensing sensor can sense the intensity of light in the exterior of the mask shell.

The above welding mask with a sensor lamp, further comprising a power supply, wherein the power supply is a rechargeable battery or a non-rechargeable battery.

The above welding mask with a sensor lamp, wherein the mechanical switch is a knob switch or a toggle switch.

The above welding mask with a sensor lamp, wherein the mechanical switch is provided with an anti-skid slot.

The above welding mask with a sensor lamp, wherein the filter module is also provided with an arc sensor, and the arc sensor is connected to the sensing module.

The above welding mask with a sensor lamp, wherein the sensing module is provided with a solar power utilization member.

The positive effects of the technical schemes are as follows: 1. the arrangement that the head lamp is provided on the filter module in the mask shell and is connected with the sensing module makes it possible to achieve the result: the head lamp will be turned on automatically in the environment with poor lighting, while turned off in the case of bright light, thereby effectively saving electric energy and making full use of resources in an more reasonable way; moreover, the sensing module is also provided with the solar power utilization member, having the ability to generate electric power through solar power, and to generate electric power by absorbing the bright light in welding, it is therefore more efficient in energy saving and environmental protection; 2. both the sensing module and the mechanical switch with a plurality of gears are provided, the welding mask can be more easily adapted to different environment, and it is possible to choose different gears depending on the environment, moreover, it is ensured that the head lamp cannot be turned on when the mechanical switch is off, and the arrangement of the plurality of gears prevents the head lamp being turned on automatically when not in use, therefore, higher security can be obtained; 3. since the head lamp is embedded in the lens module, degree of modularization is higher, and space is saved a lot; 4. arc sensor provided herein can assist the sensing module in sensing whether light is strong or weak, so that the accuracy of sensing is higher, and the intelligence level is improved further.

### BRIEF DESCRIPTION OF THE DRAWINGS

The accompanying drawings, together with the specification, illustrate exemplary embodiments of the present disclosure, and, together with the description, serve to explain the principles of the present disclosure.

FIG. 1 is a structure diagram of an embodiment of a welding mask with a sensor lamp according to the present disclosure.

FIG. 2 is a structure diagram of a view of a welding mask according to a preferred embodiment of the present disclosure.

In the accompanying drawings: 1 mask shell; 11 filter window; 3 head lamp; 4 mechanical switch; 41 anti-skid slot; 5 arc sensor; 6 sensing module.

### DETAILED DESCRIPTION

The present disclosure will now be described more fully hereinafter with reference to the accompanying drawings, in which exemplary embodiments of the disclosure are shown. This disclosure may, however, be embodied in many different forms and should not be construed as limited to the embodiments set forth herein. Rather, these embodiments are provided so that this disclosure will be thorough and complete, and will fully convey the scope of the disclosure to those skilled in the art. Like reference numerals refer to like elements throughout.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” or “includes” and/or “including” or “has” and/or “having” when used herein, specify the presence of stated features, regions, integers, steps, operations, elements, and/or components, but do not preclude the presence or addition of one or more other features, regions, integers, steps, operations, elements, components, and/or groups thereof.

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

As used herein, “around”, “about” or “approximately” shall generally mean within 20 percent, preferably within 10 percent, and more preferably within 5 percent of a given value or range. Numerical quantities given herein are approximate, meaning that the term “around”, “about” or “approximately” can be inferred if not expressly stated.

As used herein, the term “plurality” means a number greater than one.

Hereinafter, certain exemplary embodiments according to the present disclosure will be described with reference to the accompanying drawings.

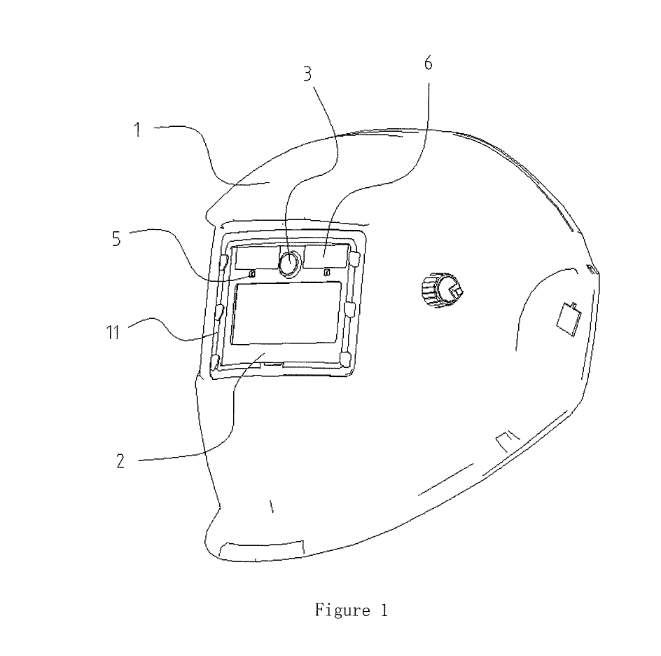

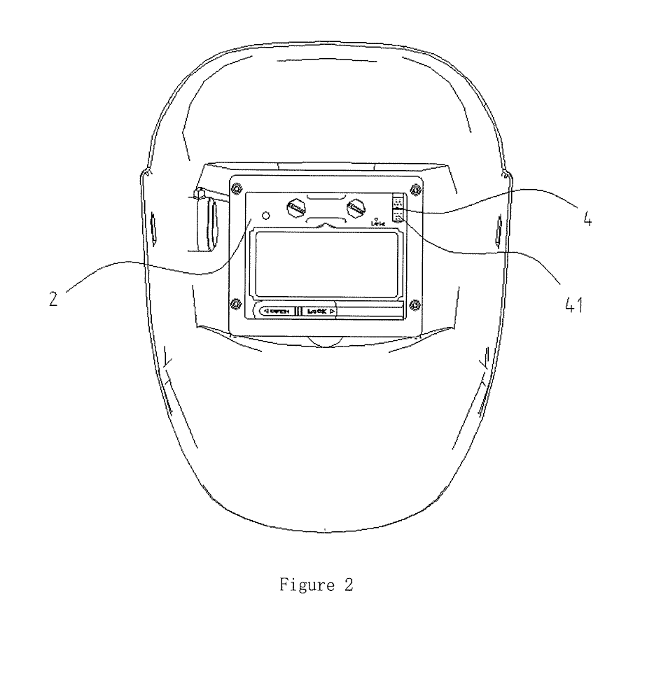

FIG. 1 is a structure diagram of an embodiment of a welding mask with a sensor lamp according to the present disclosure. FIG. 2 is a structure diagram of a view of a welding mask according to a preferred embodiment of the present disclosure. As shown in FIGS. 1 and 2, the welding mask with the sensor light provided in the embodiment comprises: a mask shell 1, a filter window 11, a filter module 2, a head lamp 3, a mechanical switch 4, an anti-skid slot 41, an arc sensor 5, and a sensing module 6.

Specifically, the mask shell 1 comprises a face portion and two side portions, and the face portion is just in front of eyes of an operator when in use, while the two side portions are disposed on either side of the face respectively, covering a face of the operator so as to prevent damage to the operator during welding.

Specifically, the filter window 11 is provided on the face portion of the mask shell 1, and the filter window 11 is arranged in an area in front of the human eyes and an area in front of and below the human eyes, and the filter window can be rectangular for easy installation of the filter module. The filter module 2 is provided in the filter window 11, and the filter module 2 is embedded in the filter window 11.

Specifically, the filter module 2 comprises a see-through lens and a light-filtering component, the lens being disposed in the face portion of the mask shell and in the area where facing towards the human eyes for easy observation of the welding operation.

Specifically, the head lamp 3 is provided in a center position above the lens of the filter module 2, the head lamp 3 is embedded in the filter module 2, and the head lamp 3 is arranged on the side facing away the face of the operator, and is connected to a circuit board of the filter module, which guarantees light emitted from the head lamp 3 can be directed on the operator at its maximum and be observed by the operator, therefore improving the quality of the weldment.

Specifically, a power supply is provided in an area below the lens on the filter module 2, and the power supply is connected onto a control switch, and connected to the head lamp 3 through the control switch, providing electric power to the head lamp 3.

More specifically, the control switch includes a mechanical switch 4 and a sensing module 6, and both the mechanical switch 4 and the sensing module 6 are connected to the circuit board of the filter module, and both the mechanical switch 4 and the sensing module 6 can switch the head lamp 3. The mechanical switch 4 is provided with a plurality of gears. One of the gears is a normally-off gear, another one of the gears is normally-on gear, another one of the gears is the sensing gear, and the sensing gear is connected to the sensing module 6. When selecting the gear of the mechanical switch 4, the normally-off gear is the lamp-off position of the head lamp 3, that is, the head lamp 3 is always in a off state when in the normally-off gear. The normally-on gear is the lamp-on position of the head lamp 3, that is, the head lamp 3 is always in an on state when in the normally-on gear; the sensing gear is sensing on and off position of the head lamp 3; when the mechanical switch 4 is in the sensing gear, the head lamp 3 turns on or off based on the light intensity of the external light sensed by the sensing module 6, The arrangement of the plurality of gears effectively ensures the reasonable work of the head lamp 3, thus it boasts stronger adaptability and higher safety.

More specifically, the mechanical switch 4 is arranged on one of the two side portions of the mask shell 1, or the mechanical switch 4 is arranged on the filter module and located on the face portion of the mask shell, which is convenient for the operator to turn on and off the head lamp manually.

More specifically, the sensing module 6 comprises a light sensing sensor and a corresponding control circuit, the light sensing sensor can sense the light intensity in the space on the mask shell 1 at a same side of the head lamp 3, and send the sensing signal to the controller. The controller controls the sensing module 6 to close when the light is weak, and the controller controls the sensing module 6 to be turned off when the light is strong, so as to control the on and off of the head lamp based on the light intensity in the external space, whereby it achieves a higher level of automation and energy-saving outcome.

More specifically, the sensing module 6 is also provided with a solar power utilization member capable of absorbing light for energy supply, thereby achieving better energy saving and environmental protection.

More specifically, the filter module 2 is further provided with an arc sensor 5, and the arc sensor 5 is also connected to the sensing module 6. The arc sensor 5 can sense stronger arc during welding, and further assist the sensing module 6 in controlling the on and off of the head lamp 3, therefore, it is more intelligent and better in energy saving.

As a preferred embodiment, the mechanical switch 4 is a knob switch or a toggle switch, and the mechanical switch 4 is provided with an anti-skid slot 41, which is convenient for the user to switch on and off the mechanical switch 4, and the operation is more convenient and reliable.

As a preferred embodiment, the filter window 11 is rectangular, facilitating the installation and disassembly of the lens module 2, and a rectangular sensing module 2 is provided, which is better to fit the power supply and the lens, and also it is more advantageous for the space layout.

As a preferred embodiment, the power supply is a dry battery or a rechargeable battery, which can be selected depending on the demand, having better adaptability.

When welding under poor lighting conditions, the operator shall select gear of the mechanical switch 4, if a long-time direct lighting is required, the normally-on gear of the mechanical switch 4 shall be selected; if it is desired to automatically select lighting according to the light intensity, the sensing gear of the mechanical switch 4 shall be selected. When selecting the sensing gear, the operator shall manually switch the mechanical switch 4 to the sensing gear first, at this point, the light sensing sensor detects a signal representing that the external light is weak, and transmits the signal to the controller, then the controller controls the head lamp 3 to start work; since the welding arc occurring during welding will produce strong and dazzling light, then both the light sensing sensor and the arc sensor 5 can detect a signal representing that the external light is strong, and transmit the signal to the controller, then the controller turns the sensing module 6 off so as to turn off the head lamp 3, whereby automatic shift is more in line with the welding environment, and the power energy consumption is lower. After the welding operation is completed, the operator shall manually switch the mechanical switch 4 to the normally-off gear, so that keep the circuit of the head lamp 3 disconnected and make the operation process simpler.

The welding mask with the sensor lamp provided by the present embodiment comprises a mask shell 1 covering a human face, a filter module 2 arranged on the mask shell 1, a head lamp 3 arranged on the filter module 2, and a control switch connected to the head lamp 3, and the control switch comprises a mechanical switch 4 and a sensing module 6; the head lamp 3 is provided on the filter module 2, so that lighting will be provided for operator in poor lighting environment, then the welding quality can be guaranteed; the control switch comprises the mechanical switch 4 and the sensing module 6, and the mechanical switch 4 has a plurality of gear selections, whereby selection in relation to normally-on, normally-off and sensor circuit is achieved, which boasts higher security, in addition, the head lamp 3 can be turned off automatically when strong light is observed during welding, achieving a higher level of automation and a better energy saving; and the head lamp 3 is provided on the filter module 2, whereby it achieves a higher level of modularization, and space utilization is more reasonable.

The foregoing is only the preferred embodiments of the invention, not thus limiting embodiments and scope of the invention, those skilled in the art should be able to realize that the schemes obtained from the content of specification and figures of the invention are within the scope of the invention.

What is claimed is:

1. A welding mask with a sensor lamp, comprising: a mask shell, a filter module, a head lamp and a control switch, wherein the filter module is arranged on the mask shell, the head lamp is embedded in the filter module and is connected to a circuit board of the filter module; wherein,
the control switch comprises a mechanical switch and a sensing module, and both the mechanical switch and the sensing module are connected to the circuit board of the filter module.

2. The welding mask with a sensor lamp of claim 1, wherein: the mask shell comprises a face portion and two side portions, and the face portion is just in front of eyes of an operator when in use, while the two side portions are disposed on either side of the face portion respectively.

3. The welding mask with a sensor lamp of claim 2, wherein: the face of the mask shell is provided with a filter window, and the filter window is rectangular.

4. The welding mask with a sensor lamp of claim 1, wherein: the mechanical switch is provided with a plurality of gears, one of the plurality of gears is a normally-off gear and another one of the plurality of gears is a normally-on gear, and another one of the plurality of gears is a sensing gear, and the mechanical switch in the sensing gear is connected to the sensing module.

5. The welding mask with a sensor lamp of claim 1, wherein: the sensing module is embedded in the filter module, and the sensing module further comprises a light sensing sensor and a corresponding control circuit, wherein the light sensing sensor can sense the intensity of light in the exterior of the mask shell.

6. The welding mask with a sensor lamp of claim 1, further comprising: a power supply, wherein the power supply is a rechargeable battery or a non-rechargeable battery.

7. The welding mask with a sensor lamp of claim 1, wherein: the mechanical switch is a knob switch or a toggle switch.

8. The welding mask with a sensor lamp of claim 7, wherein: the mechanical switch is provided with an anti-skid slot.

9. The welding mask with a sensor lamp of claim 1, wherein: the filter module is also provided with an arc sensor, and the arc sensor is connected to the sensing module.

10. The welding mask with a sensor lamp of claim 1, wherein: the sensing module is provided with a solar power utilization member.
